(19) United States
(12) Patent Application Publication
(10) Pub. No.: US 2022/0139561 A1
(43) Pub. Date: May 5, 2022

(54) Device and Method for Determining the Likelihood of a Patient Having a Clinical Event or a Clinically Silent Event Based on Ascertained Physiological Parameters

(71) Applicants: Marja Pauliina Margolis, Coral Gables, FL (US); Geoffrey Vince, Poway, CA (US); Anuja Nair, Bedford, MA (US)

(72) Inventors: Marja Pauliina Margolis, Coral Gables, FL (US); Geoffrey Vince, Poway, CA (US); Anuja Nair, Bedford, MA (US)

(73) Assignee: VOLCANO CORPORATION, Rancho Cordova, CA (US)

(21) Appl. No.: 12/890,633

(22) Filed: Sep. 25, 2010

Related U.S. Application Data
(60) Provisional application No. 61/246,043, filed on Sep. 25, 2009.

(51) Int. Cl. G16H 50/30; A61B 5/02; A61B 5/00; A61B 8/12; A61B 8/08

(52) U.S. Cl. CPC G16H 50/30; A61B 5/02007; A61B 8/0891; A61B 8/12; A61B 5/7275

(57) ABSTRACT

Using a patient's physiological parameters ascertained using standard intravascular ultrasound (IVUS) in combination with virtual histology (VH), these physiological parameters are evaluated to predict whether the patient's physiology has an increased risk of producing a Clinical Event or a Silent Clinical Event. In one embodiment the following three physiological parameters: the Plaque Burden; the Minimum Lumen Area; and whether a VH-TCFA or multiple VH-TCFAs are present are compared to target values and ranges. A concurrence of the values for the physiological parameters falling within the target ranges indicates that a patient's physiology has an increased risk of producing a Clinical Event or a Silent Clinical Event, respectively. In other embodiments, combinations of any two of the three physiological parameters listed above are compared to target values and ranges. A concurrence of the values for two of the physiological parameters falling within the target ranges indicates that a patient's physiology has an increased risk of producing a Clinical Event. In still other embodiments, any of the three physiological parameters listed above are compared to target values and ranges. Any of the values of the physiological parameters falling within the target ranges indicates that a patient's physiology has an increased risk of producing a Clinical Event.

This application claims the benefit pursuant to 35 U.S.C. § 119(e) of U.S. Provisional Patent Application No. 61/246,043, filed Sep. 25, 2009, all of which is incorporated herewith in its entirety.

### BACKGROUND OF THE INVENTION

### 1. Field of the Invention

This invention relates to improved devices and methods for treating atherosclerosis and more particularly to devices and methods for predicting clinical outcomes for patients based on an evaluation of ascertained physiological parameters related to atherosclerosis.

### 2. Description of Related Art

Coronary Artery Disease (“CAD”) is the most common type of heart disease and is the leading cause of death in the United States for both men and women. CAD is the manifestation of a condition where plaque builds up inside the coronary arteries (the arteries that supply oxygen-rich blood to the heart). Plaque is made up of fat, cholesterol, calcium, and other substances found in the blood and as a result of inflammation in artery walls. When plaque builds up in the arteries, the condition is called atherosclerosis. The buildup of plaque narrows the arteries and reduces blood flow to the heart muscle. Because plaque narrows the arteries, it also makes it more likely that blood clots will form in the arteries. These blood clots can partially or completely block blood flow.

Also, when coronary arteries are narrowed or blocked, oxygen-rich blood can't reach the heart muscle. This can cause a condition called angina or even result in a heart attack. Angina is chest pain or discomfort that occurs when not enough oxygen-rich blood is flowing to an area of the heart muscle. A heart attack occurs when blood flow to an area of your heart muscle is completely blocked. This prevents oxygen-rich blood from reaching that area of heart muscle and, as a result, the oxygen-starved area of the heart dies. Over time, CAD can weaken the heart muscle and lead to heart failure and arrhythmias. Heart failure is a condition where the heart is weakened and as a result can't pump an adequate amount of blood throughout the body.

As mentioned above, atherosclerosis is the condition where plaque builds up in the arteries. Besides just narrowing the arteries, atherosclerosis is a chronic inflammatory disorder. This disorder involves vascular, metabolic and immune systems and manifests itself in both local and systemic ways. In addition, the plaque itself has considerable variability in its morphology (composition of the plaque). The main plaque components can be characterized as being fibrotic, fibrous with higher lipid or fat content, dense calcium or necrotic core. It is common to have more than one or even all of these plaque components together in a single lesion.

There are several physiological and anatomical parameters related to coronary arteries and the buildup of plaque. Plaque Burden is one of these anatomical parameters. Plaque Burden is defined as: ((vessel area−lumen area)/vessel area)*100 and is expressed as a percentage. The vessel area is the cross-sectional area of the blood vessel not taking into account any buildup of plaque. The lumen area is the cross-sectional area of the blood vessel taking into account the buildup of plaque so that the lumen area is the area that is available for blood flow. For example, if the vessel area is 5 mm² and there is sufficient plaque buildup to produce a lumen area of 3 mm² the resulting Plaque Burden is 40% ((5 mm²−3 mm²)/5 mm²)*100. A clinically useful limit for the Plaque Burden is about 70%. This “70%” figure is recognized in the art as an amount indicating an intermediate lesion. A Plaque Burden of about 55% is also recognized in the art as the boundary of an insignificant lesion.

Another parameter of interest is the Minimum Lumen Area “MLA”. The Minimum Lumen Area is essentially the smallest lumen cross-sectional area in an area of interest along the length of the artery. An area of interest is typically the section of artery that is being imaged. A clinically useful limit for the Minimum Lumen Area is about 4 mm². Minimum Lumen Area equal to or less than about 4 mm² are considered small and hence likely to reduce flow of blood (oxygen) to heart muscle.

A particularly risky combination of plaque elements is known as a thin-cap fibroatheroma (“TCFA”). TCFA is a plaque lesion having a thin coating of fibrous tissue covering a large necrotic core. The thin coating is between the necrotic core and the lumen of the artery. What makes a TCFA troubling is that if the thin fibrous coating (the “thin cap”) ruptures, the underlying material of the necrotic core is then free to be discharged into the bloodstream where it can move downstream becoming attached to a narrow section of the artery or to another plaque lesion and result in thrombus or blot clot formation thereby partially or totally occluding the flow of blood through that artery. When there is already a narrowing in the artery, there is a higher likelihood of an undesirable event occurring should the TCFA rupture and discharge necrotic core into the artery.

Further, when a TCFA ruptures there is often a build up of thrombus at the rupture site itself. This buildup of thrombus also narrows the lumen of the artery. This is particularly problematic where there has been a series of TCFA ruptures at a particular site so that there is an accumulation of thrombus which may significantly narrow or even block the Minimum Lumen Area of the coronary artery. As a result, where the MLA is narrow, a TCFA rupture and the corresponding buildup of thrombus at that location is also more likely to produce an undesirable event.

The definition of a TCFA given above is a pathological definition. However, this pathological definition is not practical for VH-IVUS imaging since the current resolution for VH-IVUS is about 200 microns. Since a TCFA is usually pathologically defined as having a cap thickness of about 65 microns, this cap thickness would not be detectable by current VH-IVUS. As a result, a VH-TCFA is defined as a fibroatheroma where the necrotic core is equal to or exceeds 10% of the plaque with the appearance of the necrotic core being in direct contact with the vessel lumen for over a 30 degree arc from the center of the lumen for at least three consecutive VH-IVUS images along the length of the artery.

An undesirable event that may occur due to the narrowing or even occlusion of a coronary artery is a so-called Clinical Event. “Clinical Event” could be defined as the patient's death, the patient having a heart attack or the patient having unstable or increasing angina within three years. As a result, it would be highly desirable to be able to detect physiology likely to produce Clinical Events in order to prophylactically administer therapeutic or preventative care to prevent a Clinical Event from occurring.

Another undesirable event that may occur is a so-called Silent Clinical Event. A “Silent Clinical Event” may occur when the plaque ruptures. When a plaque ruptures, thrombus (the accumulation of red blood cells) typically occurs around and downstream from the rupture site. This build up of thrombus at the site of or downstream from the rupture causes a narrowing of the vessel lumen (i.e., a diminishing of the Minimum Lumen Area near the site of the rupture) but not in an amount that manifests itself in a Clinical Event. Even though this narrowing does not manifest itself in a clinically detectable manner (due to not blocking a sufficiently large amount of the coronary artery lumen), this narrowing does makes this site particularly vulnerable to additional ruptures or further narrowing or even occlusion. As a result, although a Silent Clinical Event is not a condition severe enough to warrant classification as a Clinical Event, a Silent Clinical Event is a condition with an increased risk for eventually manifesting itself in a Clinical Event. As a result, it would also be highly desirable to be able to detect physiology producing Silent Clinical Events as well in order to prophylactically administer therapeutic or preventative care to prevent a Clinical Event from occurring.

Intravascular ultrasound (“IVUS”) combined with a technique called virtual histology (“VH”) has been particularly successful in recognizing the morphology of plaque in vivo (i.e., the amount and composition of plaque in the patient's body). The following systems for detecting and characterizing plaque using IVUS with VH are disclosed in U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

FIG. 1 illustrates a typical tissue-characterization system 10 using IVUS and VH (so called “VH-IVUS”). In the typical tissue-characterization system 10, an intravascular ultrasound (IVUS) console 110 is electrically connected to an IVUS catheter 120 and used to acquire RF backscattered data (i.e., IVUS data) from a blood vessel. The VH-IVUS console 110 typically includes a computing device 130 comprising a database 134 and a characterization application 132 electrically connected to the database 134 and adapted to receive IVUS data from the IVUS console 110 or directly from a transducer 122. Specifically, a transducer 122 is attached to the end of the catheter 120 and is carefully maneuvered through a patient's arteries to a point of interest along the artery. The transducer is then pulsed to acquire high-frequency sonic echoes or backscattered signals reflected from the tissue of the vascular object. Because different types and densities of tissue absorb and reflect the ultrasound pulse differently, the reflected data (i.e., IVUS data) is used to image the vascular object. In other words, the IVUS data can be used (e.g., by the IVUS console 110 or a separate computing device 130) to create an IVUS image. An exemplary IVUS image 20 is shown in FIG. 2, where the light and dark regions indicate different tissue types and/or densities. It should be appreciated that the IVUS console 110 depicted herein is not limited to any particular type of IVUS console, and includes all ultrasonic devices known to those skilled in the art (e.g., a Revolution® or EagleEye® IVUS catheter used in conjunction with an s5™ IVUS imaging system, all of which are sold by Volcano Corporation of San Diego, Calif.). It should further be appreciated that the IVUS catheter 120 depicted herein is not limited to any particular type of catheter, and includes all ultrasonic catheters known to those skilled in the art. Thus, for example, a catheter having a single transducer (e.g., adapted for rotation) or an array of transducers (e.g., circumferentially positioned around the catheter or longitudinally along the catheter 120) can be used with the typical tissue-characterization system 10.

It should be appreciated that the database 134 depicted herein includes, but is not limited to, RAM, cache memory, flash memory, magnetic disks, optical disks, removable disks, SCSI disks, IDE hard drives, tape drives and all other types of data storage devices (and combinations thereof, such as RAID devices) generally known to those skilled in the art. It should further be appreciated that the characterization application 132, as depicted and discussed herein, may exist as a single application or as multiple applications, locally and/or remotely stored. It should also be appreciated that the number and location of the components depicted in FIG. 1 do not limit a typical tissue-characterization system 10 but are merely provided to illustrate a typical tissue-characterization system 10. Thus, for example, a computing device 130 having a plurality of databases 134 or a remotely located characterization application 132 (either in part or in whole) or any combination of these may also be found in a typical tissue-characterization system 10.

In one embodiment of a typical tissue-characterization system 10, the characterization application 132 is adapted to receive and store characterization data (e.g., tissue type, etc.). The characterization data was determined prior to using the tissue—characterization system 10 as follows. After a specimen vascular object has been interrogated (e.g., IVUS data has been collected), a histology correlation is prepared. In other words, the specimen vascular object is dissected or cross-sectioned for histology. In one method of producing characterization data, the cross-section is previously marked, for example with a suture, so that the histology can be corresponded to a portion of the IVUS image. The cross-section is then prepared with a fixing and staining process that is well known in the art. The staining process allows a clinician to identify a tissue type(s), or a chemical(s) found within (e.g., a chemical corresponding to a particular tissue type, etc.). The identified tissue type or types is then correlated to the IVUS data as will be explained below.

It should be appreciated that there may be many methods used to identify or characterize the cross-sectional object as is well understood in the art besides the method just described. Thus, any identification/characterization method generally known to those skilled in the art may be used to characterize tissue. The identified tissue type or characterization (i.e., characterization data) is then provided to the characterization application 132. In one embodiment, as shown in FIG. 1, the characterization data is provided via an input device 140 electrically connected to the computing device 130. The characterization data is preferably then stored in the database 134. It should be appreciated that the input device depicted herein includes, but is not limited to, a keyboard, a mouse, a scanner and all other data-gathering and/or data-entry devices generally known to those skilled in the art. It should further be appreciated that the term tissue type or characterization, as these terms are used herein, include, but are not limited to, fibrous tissues, fibro-lipidic tissues, calcified necrotic tissues, calcific tissues, collagen compositions, cholesterol, thrombus, compositional structures (e.g., the lumen, the vessel wall, the medial-adventitial boundary, etc.) and all other identifiable characteristics generally known to those skilled in the art.

In one method of characterizing tissue, the characterization application 132 is adapted to create a VH image where specifically characterized tissue is associated with its corresponding region on an IVUS image. Specifically, digitized data is provided to the characterization application 132 (e.g., via the IVUS catheter 120 where the digitized data corresponds to the physiology of the patient's blood vessel. The characterization application 132 uses data from the database 134 to characterize tissue corresponding to the digitized data from the IVUS catheter 120 and create a VH image (i.e., a digital image or outline that substantially corresponds to the vascular object and where specific tissue types are identified). A region of interest (ROI) on the VH image can then be identified by the operator. Preferably, the ROI is characterized by the characterization data, as previously provided, and may be the entire VH image or a portion thereof. The characterization application 132 is then adapted to identify a corresponding region (e.g., x,y coordinates, etc.) on the IVUS image where the corresponding region may be specifically identified (e.g., by coloring the corresponding region with a pre-determined color).

In view of the foregoing, what is needed is an effective device and method for alerting a healthcare provider with an indication that a patient's physiology makes the patient more likely to experience a Clinical Event or a Silent Clinical Event.

### SUMMARY OF THE INVENTION

In accordance with embodiments of this invention, a patient's physiological parameters are ascertained using standard intravascular ultrasound (IVUS) in combination with virtual histology (VH) and then these physiological parameters are evaluated to predict whether the patient's physiology is at clinically significant increased risk to produce a Clinical Event or a Silent Clinical Event compared to the population at large. In particular, in one embodiment the following three physiological parameters: the Plaque Burden; the Minimum Lumen Area; and whether a VH-TCFA or multiple VH-TCFAs are present are compared to target values and ranges. A concurrence of the values for the physiological parameters falling within the target ranges indicates that a patient's physiology is at an increased risk to produce a Clinical Event or a Silent Clinical Event, respectively than the population at large. In other embodiments, combinations of any two of the three physiological parameters listed above are compared to target values and ranges. A concurrence of the values for two of the physiological parameters falling within the target ranges indicates that a patient's physiology is also at an increased risk to produce a Clinical Event than the population at large. In still other embodiments, any of the three physiological parameters listed above are compared to target values and ranges. Any of the values of the physiological parameters falling within the target ranges indicates that a patient's physiology is at an increased risk to produce a Clinical Event than the population at large.

It is an object of this invention in one or more embodiments to ascertain whether a patient's physiology puts the patient at an increased risk of experiencing a Clinical Event.

It is an object of this invention in one or more embodiments to ascertain whether a patient's physiology puts the patient at an increased risk of experiencing a Silent Clinical Event.

The invention will be described hereafter in detail with particular reference to the drawings. Throughout this description, like elements, in whatever embodiment described, refer to common elements wherever referred to and referenced by the same reference number. The characteristics, attributes, functions, interrelations ascribed to a particular element in one location apply to that element when referred to by the same reference number in another location unless specifically stated otherwise.

### BRIEF DESCRIPTIONS OF THE DRAWINGS

FIG. 1 is a schematic view of a typical tissue-characterization system using IVUS and VH.

FIG. 2 is an exemplary IVUS image.

FIG. 3 is an exemplary VH image.

FIG. 4 is a schematic view of an embodiment of the present invention applied to the tissue-characterization system of FIG. 1.

FIG. 5 is a flowchart showing the algorithm of one embodiment of the invention.

FIG. 6 is a Venn diagram associated with the algorithm of FIG. 5 showing the concurrence of conditions needed to produce the condition “Increased Risk of Clinical Event.”

FIG. 7 is a flowchart showing the algorithm of another embodiment of the invention.

FIG. 8 is a Venn diagram associated with the algorithm of FIG. 7 showing the concurrence of conditions needed to produce the condition “Increased Risk of Silent Clinical Event.”

FIG. 9 is a flowchart showing the algorithm of another embodiment of the invention.

FIG. 10 is a Venn diagram associated with the algorithm of FIG. 9 showing the concurrence of conditions needed to produce the condition “Increased Risk of Clinical Event.”

FIG. 11 is a flowchart showing the algorithm of another embodiment of the invention.

FIG. 12 is a Venn diagram associated with the algorithm of FIG. 11 showing the concurrence of conditions needed to produce the condition “Increased Risk of Clinical Event.”

FIG. 13 is a flowchart showing the algorithm of another embodiment of the invention.

FIG. 14 is a Venn diagram associated with the algorithm of FIG. 13 showing the concurrence of conditions needed to produce the condition “Increased Risk of Clinical Event.”

FIG. 15 is a flowchart showing the algorithm of another embodiment of the invention.

FIG. 16 is a Venn diagram associated with the algorithm of FIG. 15 showing the condition needed to produce the condition “Increased Risk of Clinical Event.”

FIG. 17 is a flowchart showing the algorithm of another embodiment of the invention.

FIG. 18 is a Venn diagram associated with the algorithm of FIG. 17 showing the condition needed to produce the condition “Increased Risk of Clinical Event.”

FIG. 19 is a flowchart showing the algorithm of another embodiment of the invention.

FIG. 20 is a Venn diagram associated with the algorithm of FIG. 19 showing the condition needed to produce the condition “Increased Risk of Clinical Event.”

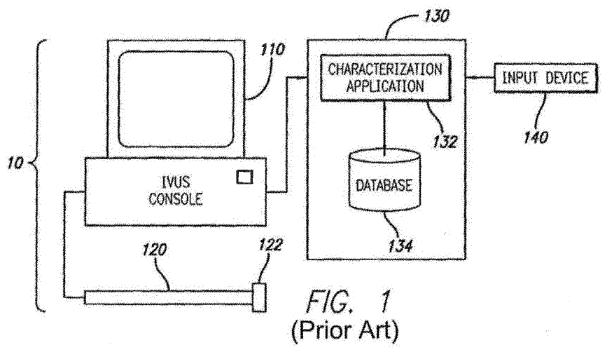

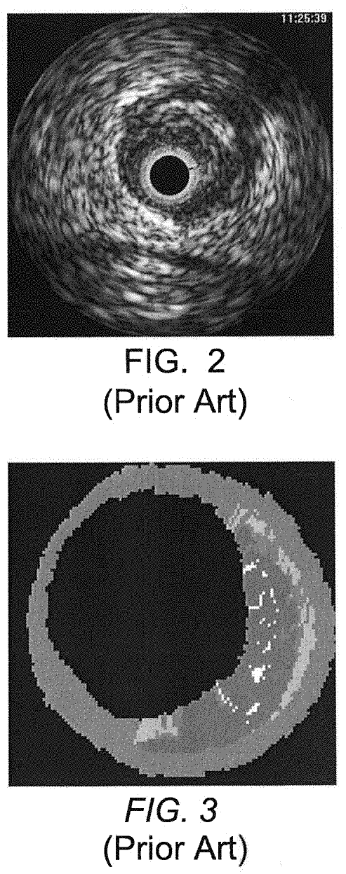

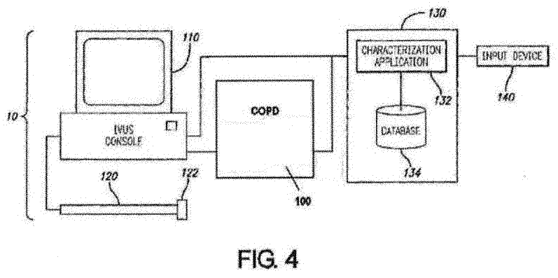

### DESCRIPTION OF THE PREFERRED EMBODIMENTS

Referring to the Figures, a Clinical Outcome Predictor Device (COPD) is shown in the Figures generally referred to by the reference number 100. The COPD 100 includes or may be an adjunct to a the tissue-characterization system 10 as described above including a console 110, IVUS catheter 120, a computing device 130 comprising a database 134 and a characterization application 132 electrically connected to the database 134 and typically run on the computing device 130. The COPD 100 is adapted to communicate, that is both receive and transmit data and information, from the IVUS console 110 or the computing device 130 or both. The COPD 100 is preferably operated on the computing device 130 but may also be operated separately and operatively connected to the console 110, computing device 130, characterization application 132 or database 134 or any combination of these.

The COPD 100 is used to determine whether a patient's physiology indicates that the patient has an increased risk of having a Clinical Event or a Silent Clinical Event compared to the population at large. By increased risk we mean that the Hazard Ratio exceeds 2.0. The Hazard Ratio is a measure of how often a particular event happens in one group compared to how often it happens in another group, over time. A Hazard Ratio of one here means that there is no difference in the occurrence of a Clinical Event or Silent Clinical Event between the two groups. A Hazard Ratio of greater than two means that occurrence of a Clinical Event or Silent Clinical Event was significantly more likely in one of the groups. Here, the control group is the population at large and the comparison group is the group with the particular physiology ascribed to each method and device described hereafter. The higher the Hazard Ratio, the more likely patient's having the particular physiology are to have a Clinical Event or Silent Clinical Event as compared to the population at large.

The tissue-characterization system 10 is used to ascertain the Plaque Burden of the patient's target coronary artery, the Minimum Lumen Area of that coronary artery and whether a VH-TCFA is present in that coronary artery according to standard processes and algorithms as is well understood by those skilled in the art. That data is typically stored in the database 134 to be used as will be described hereafter.

The COPD 100 then evaluates the ascertained Plaque Burden, Minimum Lumen Area and whether a VH-TCFA is present to determine whether the patient's physiology is at an increased risk to have a Clinical Event. In a preferred embodiment of the COPD 100, the COPD 100 is a software application run on the computing device 130.

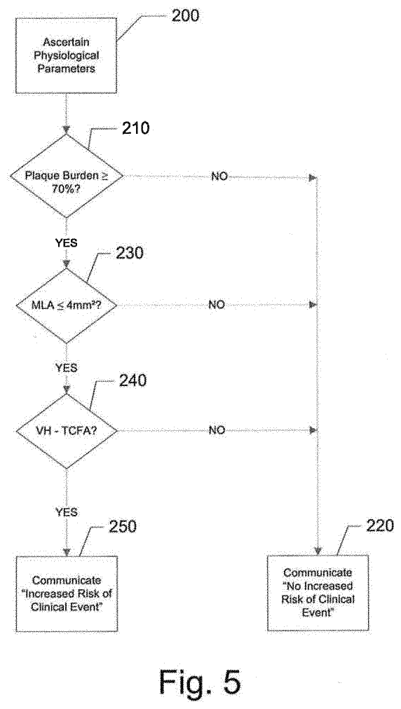

One method of performing this evaluation is by running an algorithm on the COPD 100 as will be described hereafter. FIG. 5 is a flowchart illustrating an example of the algorithm. In FIG. 5 at step 200, values for the Plaque Burden, Minimum Lumen Area and whether a VH-TCFA is present are determined by the use of the tissue-characterization system 10 as described above. The program then passes to step 210 where the ascertained value for the Plaque Burden is compared to an established or target range of Plaque Burdens. The established or target range is about 70% to 100%. This range represents a Plaque Burden where plaque substantially to completely occludes the lumen of the artery. If the ascertained Plaque Burden does not fall within this range, the program passes to step 220 where the determination that the physiological parameter is not within the target range, and thus that the patient's physiology puts the patient less at risk to have a Clinical Event, is communicated to the healthcare provider.

If the ascertained Plaque Burden falls within this target range, the program passes to step 230 where the ascertained Minimum Lumen Area of interest is compared to predetermined or target Minimum Lumen Areas. The target Minimum Lumen Area in step 230 is less than or equal to about 4 mm². This target Minimum Lumen Area represents a relatively small open lumen in the arterial section of interest. Since these coronary arteries have relatively small Minimum Lumen Areas at least at the point where there is a relatively high Plaque Burden, they are more likely to be affected by the presence of necrotic core, thrombus or other materials capable of occluding the coronary artery lumen. Coronary arteries with relatively larger Minimum Lumen Areas are less likely to be occluded in a clinically significant way by the presence of these occluding entities. If the ascertained Minimum Lumen Area does not fall within this range, the program passes to step 220.

If the Minimum Lumen Area of the coronary artery falls within the target range, the program passes to step 240 where it is determined whether a VH-TCFA is present. If a VH-TCFA is not present, as ascertained by the tissue-characterization system 10, the program passes to step 220. If a VH-TCFA is present, the program passes to step 250.

In order for the program to have progressed to step 250, all three ascertained physiological parameters have had to have values falling within the target ranges. This indicates that the patient's physiological condition has an increased risk of manifesting itself in a Clinical Event. Step 250 communicates this determination to the healthcare provider so that appropriate medical response can be undertaken.

Likewise, as mentioned above, step 220 is reached if any physiological parameter of interest does not fall within the target range. This indicates that the patient's physiological condition does not have the increased risk of manifesting itself in a Clinical Event that the concurrence of these parameters falling within their target ranges produces. As a result, this information should be communicated to the healthcare provider as well.

In the algorithm described above, the steps of comparing the Plaque Burden, Minimum Lumen Area and whether a VH-TCFA is present have been described as being performed in a specific order. However, for this embodiment of the invention, these steps may be performed in any order.

In practice, the values for the Plaque Burden, Minimum Lumen Area and whether a VH-TCFA is present vary with location along a particular artery of interest. Data for determining these values is gathered by the tissue-characterization system 10 by inserting the IVUS catheter 120 into the artery of interest and then pulling the IVUS catheter 120 back while gathering data. Each bit of gathered data is specific to a particular location along the artery of interest. In order for there to be a determination that there is an a Increased Risk of Clinical Event, for at least one location along the artery of interest, all three “targets” must be met for at least that location. In other words, for there to be a determination that the patient's physiology puts him or her at an increased risk for a Clinical Event, for at least one location along the artery of interest, the Plaque Burden must be equal to or greater than about 70%, the Minimum Lumen Area has to be less than or equal to about 4 mm² and a VH-TCFA has to be present.

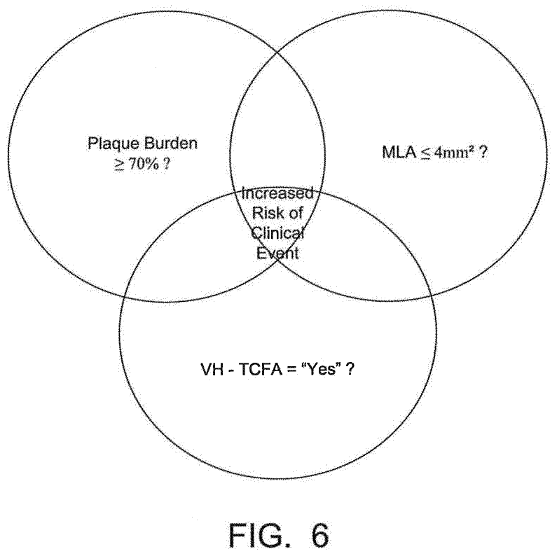

As a result, the determination of whether the patient's physiology puts him or her at an increased risk for a Clinical Event can be thought of in the context of a Venn diagram (FIG. 6) where the condition “Increased Risk of Clinical Event” is the concurrence of the Plaque Burden being equal to or greater than about 70%, the Minimum Lumen Area being less than or equal to about 4 mm² and a VH-TCFA is present. Although a specific implementation of determining whether the condition “Increased Risk of Clinical Event” has been described above, it is intended that any system for determining whether the condition “Increased Risk of Clinical Event” is present by the concurrence of these physiological parameters meeting the stated “target” ranges or values is intended to fall within the scope of the invention.

In the embodiment of the invention described above, an algorithm was used only to determine whether the patient's physiological parameters were in a state with an increased risk of manifesting itself in a Clinical Event. In an alternate embodiment of the invention, the COPD 100 is used to detect whether a patient's physiology puts the patient at an increased risk of having a Clinically Silent Event either singly or in combination with detecting physiology putting the patient at an increased risk of having a Clinical Event.

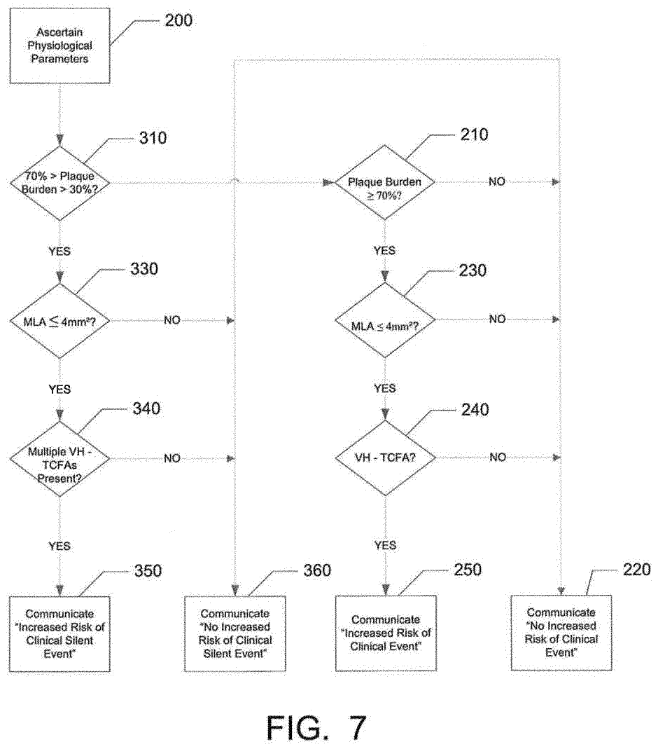

One method of performing this evaluation is by running an algorithm on the COPD 100. FIG. 7 is a flowchart illustrating such an algorithm. In FIG. 7, values for the Plaque Burden, Minimum Lumen Area and whether a VH-TCFA is present are determined by the use of the tissue-characterization system 10 in step 200. Again, these values for the Plaque Burden of the patient's target coronary artery, the Minimum Lumen Area of that coronary artery and whether a VH-TCFA is present in that coronary artery are ascertained according to standard processes and algorithms as is well understood by those skilled in the art. That data is stored in the database 134 to be used as will be described hereafter.

The program then passes to step 310 where the ascertained value for the Plaque Burden is compared to an established or target range of Plaque Burdens. The established or target range is between about 30% and about 70%. This range represents a Plaque Burden where plaque is beginning to occlude the lumen of the artery but is not yet significantly occluding the artery. If the ascertained Plaque Burden does not fall within this range, the program passes to step 210 where an analysis of whether the patient's physiology puts the patient at an increased risk of experiencing a Clinical Event is performed as described above. If, at step 210 it is determined that the patient's Plaque Burden is not equal to or greater than 70%, this indicates that the patient's Plaque Burden is equal to or less than about 30%. As a result, the program progresses from step 210 to both steps 220 and 360 as will be explained below. The outcomes of the analysis performed by steps 210, 230 and 240 in the embodiment shown in FIG. 7 is either that the patient's physiology puts the patient at an increased risk of experiencing a Clinical Event (step 250) or, as communicated at step 220, that the patient is not at an increased risk of experiencing a Clinical Event that the concurrence of parameters falling with in target values or steps 210, 230 and 240 require.

If, at step 310, the ascertained Plaque Burden falls within the 30% to 70% target range, the program passes to step 330 where the ascertained Minimum Lumen Area of the coronary artery of interest is compared to predetermined or target Minimum Lumen Area of that coronary artery. The target Minimum Lumen Area in step 330 is again less than or equal to about 4 mm². Minimum Lumen Areas larger than about 4 mm² represent relatively large coronary arteries. Since such coronary arteries have relatively large Minimum Lumen Area, they are less likely to be affected by the presence of necrotic core, thrombus or other materials capable of occluding the coronary artery lumen as compared to coronary arteries with smaller Minimum Lumen Areas as described above. If the ascertained coronary Minimum Lumen Area does not fall within this range (i.e., the Minimum Lumen Area is larger than about 4 mm²), the program passes to step 360 which indicates that the patient's physiological condition does not have the increased risk of manifesting itself in a Silent Clinical Event that the concurrence of these parameters falling within their target ranges produces.

If the Minimum Lumen Area of the coronary artery falls within the target range (i.e., the Minimum Lumen Area is smaller than about 4 mm²), the program passes to step 340 where it is determined whether multiple VH-TCFAs are present. If multiple VH-TCFAs are not present, as ascertained by the tissue-characterization system 10, the program passes to step 360. If multiple VH-TCFAs are present the program passes to step 350.

In order for the program to have progressed to step 350, all three ascertained physiological parameters have had to have values falling within the target ranges. This indicates that the patient's physiological condition is in the condition called “Silent Clinical Event.” Step 350 communicates this determination to the healthcare provider so that appropriate medical response can be undertaken.

Likewise, step 360 is reached if any physiological parameter of interest does not fall within the target range for indicating that the patient's physiology does not have the increased risk of manifesting itself in a Silent Clinical Event that the concurrence of these parameters falling within their target ranges produces. This indicates that the patient's physiological condition does not have an increased risk of manifesting itself in a Silent Clinical Event. As a result, this information should be communicated to the healthcare provider as well.

Again, in practice, the values for the Plaque Burden, Minimum Lumen Area and whether a VH-TCFA is present vary with location along a particular artery of interest. Data for determining these values is gathered by the tissue-characterization system 10 by inserting the IVUS catheter 120 into the artery of interest and then pulling the IVUS catheter 120 back while gathering data. Each bit of gathered data is specific to a particular location along the artery of interest. In order for there to be a determination that a patient's physiology puts the patient at an increased risk of Silent Clinical Event, for at least one location along the artery of interest, all three “targets” must be met. In other words, for there to be a determination that there is an increased risk of Silent Clinical Event, for at least one location along the artery of interest, the Plaque Burden must be between about 30% and about 70%, the Minimum Lumen Area has to be less than or equal to about 4 mm² and multiple VH-TCFAs have to be present.

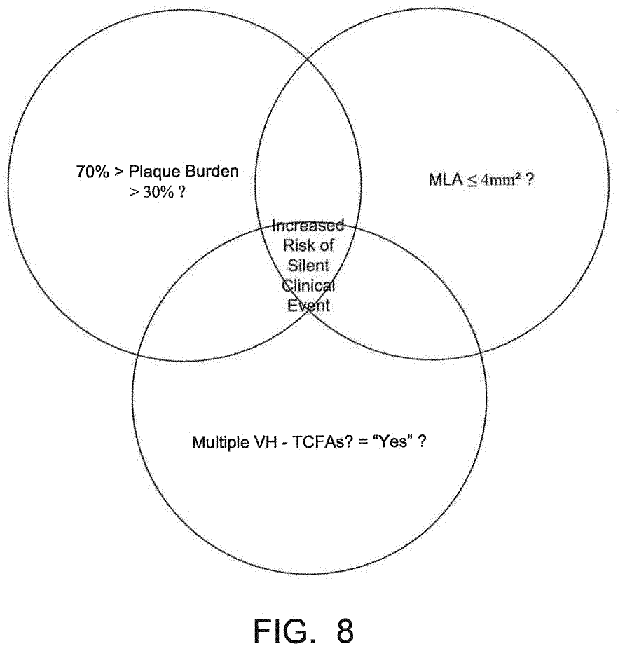

As a result, the determination of whether there is an increased risk of Silent Clinical Event can be thought of in the context of a Venn diagram (FIG. 8) where the condition “Increased Risk of Silent Clinical Event” is the concurrence of the Plaque Burden being between about 30% to about 70%, the Minimum Lumen Area being less than or equal to about 4 mm² and multiple VH-TCFAs being present. Although a specific implementation of determining whether the condition “Increased Risk of Silent Clinical Event” has been described above, it is intended that any system for determining whether the condition “Increased Risk of Silent Clinical Event” is present by the concurrence of these physiological parameters meeting the stated “target” ranges or values is intended to fall within the scope of the invention.

Further, the determination of whether there is an “Increased Risk of Silent Clinical Event” may be determined in a variant of this algorithm separately of a determination of whether there is an “Increased Risk of Clinical Event.” Determining whether there is an “Increased Risk of Silent Clinical Event” separately of a determining whether there is an “Increased Risk of Clinical Event” may easily be done by removing the steps on FIG. 7 as described above related to determining whether there is an “Increased Risk of Clinical Event.”

In the embodiments of the invention described above, a concurrence of the three physiological parameters: the Plaque Burden; the Minimum Lumen Area; and whether there is a VH-TCFA or multiple VH-TCFAs are compared to target values and ranges. As explained, a concurrence of the values for the physiological parameters falling within the target ranges indicates that a patient's physiology puts the patient at increased risk for having a Clinical Event or a Silent Clinical Event, respectively. However, it is also within the scope of the present invention for combinations of any two of the three physiological parameters listed above to be individually compared to target values and ranges.

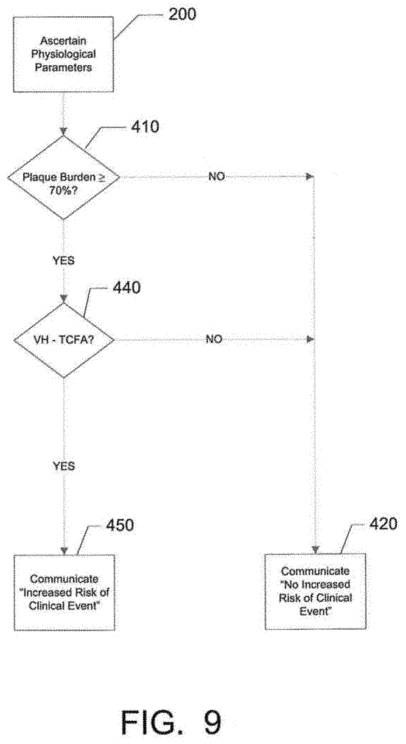

In particular, another method for determining whether there is an increased likelihood of having a Clinical Event is performed by running an algorithm on the COPD 100 as will be described hereafter. FIG. 9 is a flowchart illustrating an example of the algorithm. In FIG. 9 at step 200, values for the following patient physiological parameters: the Plaque Burden and whether a VH-TCFA is present are determined by the use of the tissue-characterization system 10 as described above. The program then passes to step 410 where the ascertained value for the Plaque Burden is compared to an established or target range of Plaque Burdens. The established or target range is about 70% to 100%. This range represents a Plaque Burden where plaque substantially to completely occludes the lumen of the artery. If the ascertained Plaque Burden does not fall within this range, the program passes to step 420 where the determination that the physiological parameter is not within the target range, and thus that the patient's physiology does not the patient at an increased risk to have a Clinical Event, is communicated to the healthcare provider.

If, at step 410, the ascertained Plaque Burden falls within this target range, the program passes to step 440 where it is determined whether a VH-TCFA is present. If a VH-TCFA is not present, the program passes to step 420. If a VF-TCFA is present, the program passes to step 450.

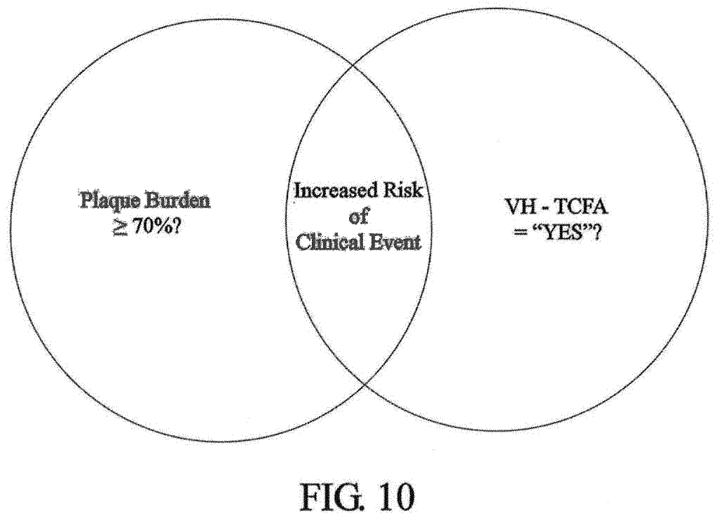

In order for the program to have progressed to step 450, both ascertained physiological parameters have had to have values falling within the target ranges (FIG. 10). This indicates that the patient's physiological condition has an increased risk of manifesting itself in a Clinical Event. Step 450 communicates this determination to the healthcare provider so that an appropriate medical response can be undertaken.

Likewise, as mentioned above, step 420 is reached if any physiological parameter of interest does not fall within the target range. This indicates that the patient's physiological condition does not have a significantly increased risk of manifesting itself in a Clinical Event. As a result, this information should be communicated to the healthcare provider as well.

In the algorithm described above, the steps of comparing the Plaque Burden and whether a VH-TCFA is present have been described as being performed in a specific order. However, for this embodiment of the invention, these steps may be performed in any order.

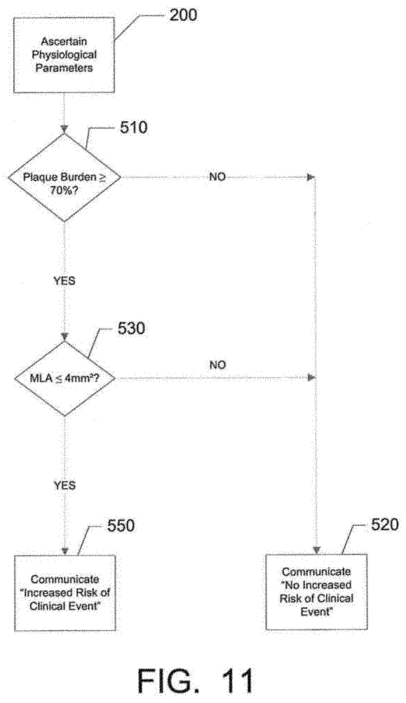

Another method for determining whether there is an increased risk of having a Clinical Event is performed by running an algorithm on the COPD 100 as will be described hereafter. FIG. 11 is a flowchart illustrating an example of the algorithm. In FIG. 11 at step 200, values for the following patient physiological parameters: the Plaque Burden and Minimum Lumen Area are determined by the use of the tissue-characterization system 10 as described above. The program then passes to step 510 where the ascertained value for the Plaque Burden is compared to an established or target range of Plaque Burdens. The established or target range is about 70% to 100%. This range represents a Plaque Burden where plaque substantially to completely occludes the lumen of the artery. If the ascertained Plaque Burden does not fall within this range, the program passes to step 520 where the determination that the physiological parameter is not within the target range, and thus that the patient's physiology does not put the patient at an increased risk to have a Clinical Event, is communicated to the healthcare provider

If the ascertained Plaque Burden falls within this target range, the program passes to step 530 where the ascertained Minimum Lumen Area of interest is compared to predetermined or target Minimum Lumen Areas. The target Minimum Lumen Area in step 530 is less than or equal to about 4 mm². This target Minimum Lumen Area represents a relatively small open lumen in the arterial section of interest. Since these coronary arteries have relatively small Minimum Lumen Areas, at least at the point where there is a relatively high Plaque Burden, they are more likely to be affected by the presence of necrotic core, thrombus or other materials capable of occluding the coronary artery lumen. Coronary arteries with relatively larger Minimum Lumen Areas are less likely to be occluded in a clinically significant way by the presence of these occluding entities. If the ascertained Minimum Lumen Area does not fall within this range, the program passes to step 520. If the Minimum Lumen Area of the coronary artery falls within the target range, the program passes to step 550.

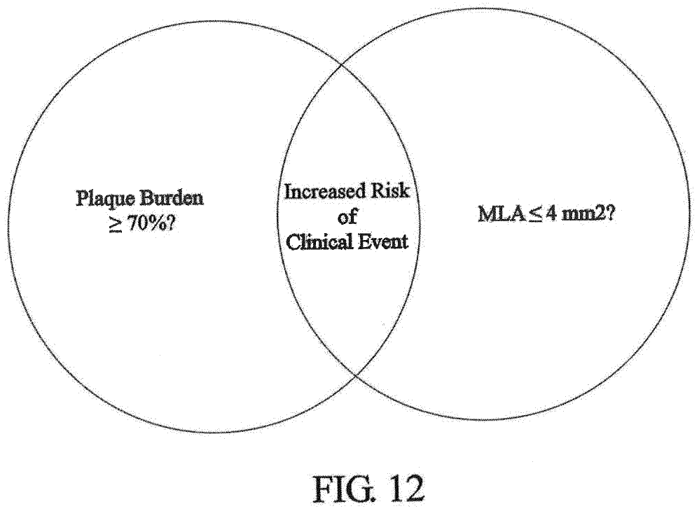

In order for the program to have progressed to step 550, both ascertained physiological parameters have had to have values falling within the target ranges (FIG. 12). This indicates that the patient's physiological condition has an increased risk of manifesting itself in a Clinical Event. Step 550 communicates this determination to the healthcare provider so that appropriate medical response can be undertaken.

Likewise, as mentioned above, step 520 is reached if any physiological parameter of interest does not fall within the target range. This indicates that the patient's physiological condition does not have put the patient at an increased risk of manifesting itself in a Clinical Event. As a result, this information should be communicated to the healthcare provider as well.

In the algorithm described above, the steps of comparing the Plaque Burden and Minimum Lumen Area have been described as being performed in a specific order. However, for this embodiment of the invention, these steps may be performed in any order.

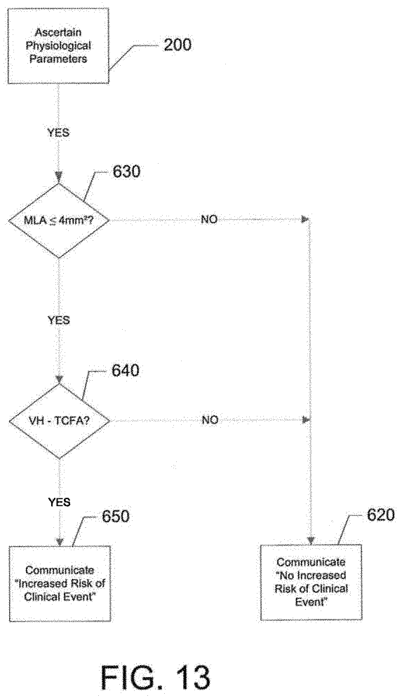

Yet another method for determining whether there is an increased likelihood of having a Clinical Event is performed by running an algorithm on the COPD 100 as will be described hereafter. FIG. 13 is a flowchart illustrating an example of the algorithm. In FIG. 13 at step 200, values for the following patient physiological parameters: the Minimum Lumen Area and whether a VH-TCFA is present are determined by the use of the tissue-characterization system 10 as described above. The program then passes to step 630 where the ascertained value for the Minimum Lumen Area is compared to an established or target range of Minimum Lumen Areas. The established or target range is about less than or equal to about 4 mm². If the ascertained Minimum Lumen Area does not fall within this range, the program passes to step 620 where the determination that the physiological parameter is not within the target range, and thus that the patient's physiology does not put the patient at an increased risk to have a Clinical Event, is communicated to the healthcare provider.

If the ascertained Minimum Lumen Area falls within this target range, the program passes to step 640 where it is determined whether a VH-TCFA is present. If a VH-TCFA is not present, the program passes to step 620. If a VH-TCFA is present, the program passes to step 650.

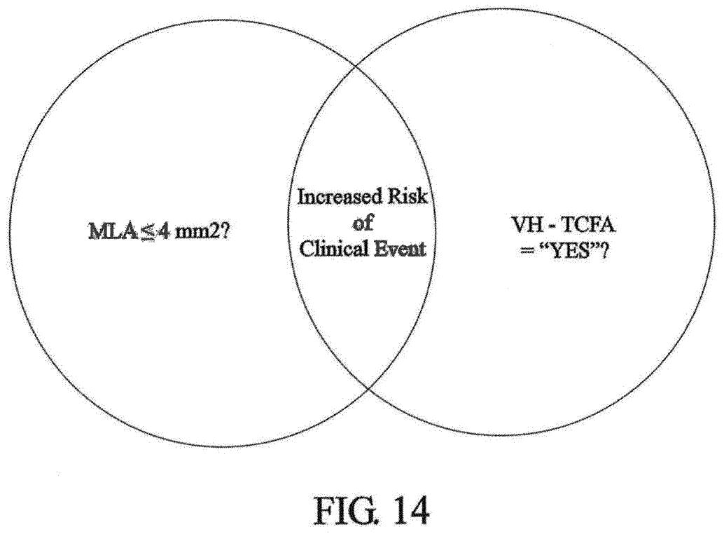

In order for the program to have progressed to step 650, both ascertained physiological parameters have had to have values falling within the target ranges (FIG. 14). This indicates that the patient's physiological condition has an increased risk of manifesting itself in a Clinical Event. Step 650 communicates this determination to the healthcare provider so that appropriate medical response can be undertaken.

Likewise, as mentioned above, step 620 is reached if any physiological parameter of interest does not fall within the target range. This indicates that the patient's physiological condition does not have a significantly increased risk of manifesting itself in a Clinical Event. As a result, this information should be communicated to the healthcare provider as well. In the algorithm described above, the steps of comparing the Minimum Lumen Area and determining whether a VH-TCFA is present have been described as being performed in a specific order. However, for this embodiment of the invention, these steps may be performed in any order.

In the embodiments of the invention described above, various combinations of two out of the three physiological parameters: the Plaque Burden; the Minimum Lumen Area; and whether a VH-TCFA or multiple VH-TCFAs are present and compared to target values and ranges and concurrences of any two of these physiological parameters with their corresponding target ranges indicates that a patient's physiology is likely to produce a Clinical Event or a Silent Clinical Event, respectively. However, it is also within the scope of the present invention for any of the three physiological parameters listed above to be compared to target values and ranges.

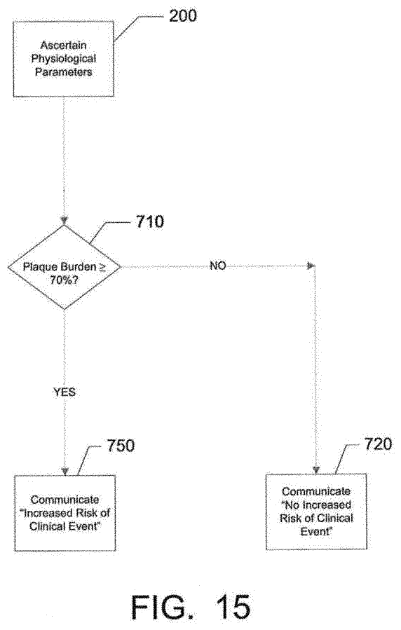

Consequently, another method for determining whether there is an increased likelihood of having a Clinical Event is performed by running an algorithm on the COPD 100 as will be described hereafter. FIG. 15 is a flowchart illustrating an example of the algorithm. In FIG. 15 at step 200, the value for the Plaque Burden is determined by the use of the tissue-characterization system 10 as described above. The program then passes to step 710 where the ascertained value for the Plaque Burden is compared to an established or target range of Plaque Burdens. The established or target range is about 70% to 100%. If the ascertained Plaque Burden does not fall within this range, the program passes to step 720 where the determination that the physiological parameter is not within the target range, and thus that the patient's physiology does not put the patient at an increased risk to have a Clinical Event, is communicated to the healthcare provider. If the ascertained Plaque Burden falls within this target range, the program passes to step 750.

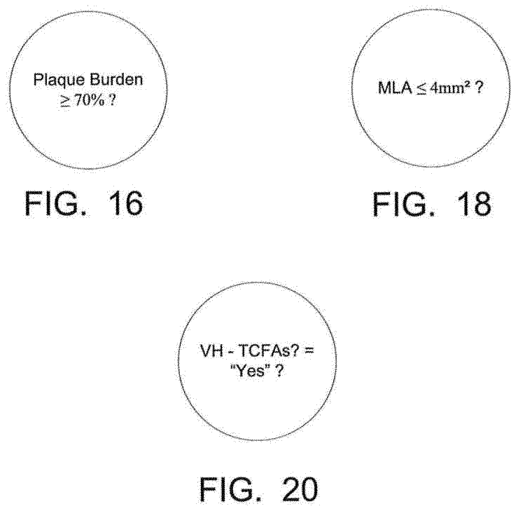

In order for the program to have progressed to step 750, the value ascertained for the Plaque Burden must have had a value falling within the target range (FIG. 16). This indicates that the patient's physiological condition does not put the patient at an increased risk of manifesting itself in a Clinical Event. Step 750 communicates this determination to the healthcare provider so that appropriate medical response can be undertaken.

Likewise, as mentioned above, step 720 is reached if the plaque burden does not fall within the target range. This indicates that the patient's physiological condition does not put the patient at an increased risk of manifesting itself in a Clinical Event. As a result, this information should be communicated to the healthcare provider as well

Yet a further method determining the increased likelihood of having a Clinical Event is performed by running an algorithm on the COPD 100 as will be described hereafter. FIG. 17 is a flowchart illustrating an example of the algorithm. In FIG. 17 at step 200, the value for the Minimum Lumen Area is determined by the use of the tissue-characterization system 10 as described above. The program then passes to step 830 where the ascertained value for the Minimum Lumen Area is compared to an established or target range of Minimum Lumen Areas. The established or target range is about less than or equal to about 4 mm². If the ascertained Minimum Lumen Area does not fall within this range, the program passes to step 830 where the determination that the physiological parameter is not within the target range, and thus that the patient's physiology does not put the patient at an increased risk to have a Clinical Event, is communicated to the healthcare provider. If the ascertained Minimum Lumen Area falls within this target range, the program passes to step 850.

In order for the program to have progressed to step 850, the Minimum Lumen Area had to have a value falling within the target ranges (FIG. 18). This indicates that the patient's physiological condition has an increased risk of manifesting itself in a Clinical Event. Step 850 communicates this determination to the healthcare provider so that appropriate medical response can be undertaken.

Likewise, as mentioned above, step 820 is reached if the Minimum Lumen Area does not fall within the target range. This indicates that the patient's physiological condition does not put the patient at an increased risk of manifesting itself in a Clinical Event. As a result, this information should be communicated to the healthcare provider as well.

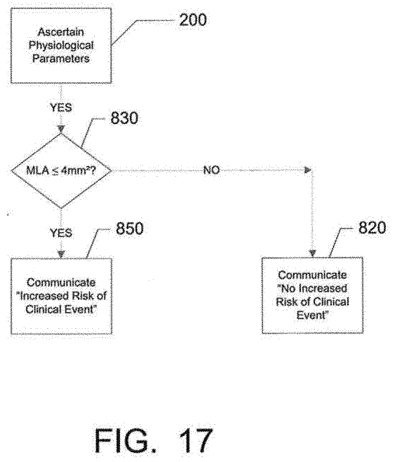

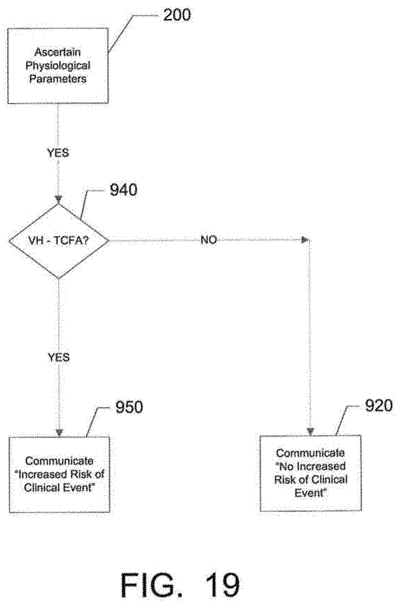

Another method of determining whether there is an increased likelihood of having a Clinical Event is performed by running an algorithm on the COPD 100 as will be described hereafter. FIG. 19 is a flowchart illustrating an example of the algorithm. In FIG. 19 at step 200, the data necessary to determine whether a VH-TCFA is present is collected by the use of the tissue-characterization system 10 as described above. The program then passes to step 940 where it is determined whether a VH-TCFA is present. If a VH-TCFA is not present, the program passes to step 920 where the determination that the physiological parameter is not within the target range, and thus that the patient's physiology does not put the patient at an increased risk to have a Clinical Event, is communicated to the healthcare provider. If a VH-TCFA is present, the program passes to step 950.

In order for the program to have progressed to step 950, a VH-TCFA must be present (FIG. 20). This indicates that the patient's physiological condition has an increased risk of manifesting itself in a Clinical Event. Step 950 communicates this determination to the healthcare provider so that appropriate medical response can be undertaken.

Likewise, as mentioned above, step 920 is reached is a VH-TCFA is not present. This indicates that the patient's physiological condition does not put the patient at an increased risk of manifesting itself in a Clinical Event. As a result, this information should be communicated to the healthcare provider as well.

Steps 250, 450, 550, 650, 750, 850 and 950 communicate to the healthcare provider that the respective parameter or parameters have all fallen within target ranges thereby indicating that the patient's physiology has an increased risk of producing a Clinical Event so that appropriate medical response can be undertaken. The communication in steps 250, 450, 550, 650, 750, 850 and 950 may take the form of a message displayed on console 110, the modifying of an image of the coronary artery displayed on the console 110 such as by appending a text or color indicator that the patient's physiology at that location on the artery is such that the patient is more likely to experience a Clinical Event, the communication of the parameter values themselves separately or as part of one of the previous messages or by any other means well within the skill of one skilled in the art to communicate such a determination.

Likewise, as mentioned above, steps 220, 420, 520, 620, 720, 820 and 920 are reached if any physiological parameter of interest does not fall within the target range. This indicates that the patient's physiological condition does not have an increased risk of manifesting itself in a Clinical Event. As a result, this information should be communicated to the healthcare provider as well. The communication in steps 220, 420, 520, 620, 720, 820 and 920 may also take the form of a message displayed on console 110, the modifying of an image of the coronary artery displayed on the console 110 such as by appending a text or color indicator that the patient's physiology at that location on the artery is such that the patient is not at an increased risk to experience a Clinical Event, the communication of the parameter values themselves separately or as part of one of the previous messages or by any other means well within the skill of one skilled in the art to communicate such a determination.

Step 350 communicates to the healthcare provider that the Plaque Burden, Minimum Lumen Area and that a VH-TCFA or multiple VH-TCFAs are present having values all falling within the target ranges of steps 310, 330 and 340 thereby indicating that the patient's physiology has an increased risk of producing a Silent Clinical Event so that appropriate medical response can be undertaken. The communication in step 350 may take the form of a message displayed on console 110, the modifying of an image of the coronary artery displayed on the console 110 such as by appending a text or color indicator that the patient's physiology at that location on the artery is such that the patient is at an increased risk to experience a Silent Clinical Event, the communication of the parameter values themselves separately or as part of one of the previous messages or by any other means well within the skill of one skilled in the art to communicate such a determination.

Further, as mentioned above, step 360 is reached if the respective physiological parameters are not within the targets of steps 310, 330 and 340, and in the embodiment shown in FIG. 7 having steps 210, 230 and 240 are not within the targets of steps 210, 230 and 240, and thus do not fall within the target range. This indicates that the patient's physiological condition does not have an increased risk of manifesting itself in a Silent Clinical Event. As a result, this information should be communicated to the healthcare provider as well. The communication in step 360 may also take the form of a message displayed on console 110, the modifying of an image of the coronary artery displayed on the console 110 such as by appending a text or color indicator that the patient's physiology at that location on the artery is such that the patient is not at an increased risk to experience a Silent Clinical Event, the communication of the parameter values themselves separately or as part of one of the previous messages or by any other means well within the skill of one skilled in the art to communicate such a determination.

Although various embodiments of the invention have been described as being executed in the operation of an algorithm by a microprocessor in the COPD 100, the algorithms could be implemented through the use of a logic or neural network. Alternately the algorithms could be established on discrete analog or digital components as part of the COPD 100.

The target range for the Plaque Burden in several of the embodiments herein has been described as being equal to or larger than about 70%. As explained above, this “70%” figure is recognized in the art as an amount indicating an intermediate lesion. A Plaque Burden of about 55% is also recognized in the art as the boundary of an insignificant lesion. Consequently, the “70%” figure used in the embodiments could be replaced with “55%” and the present invention, in the respective embodiments, would still produce a predictor of events having a Hazard Ratio exceeding 2.0. Although “55%” and “70%” have been given as boundaries of clinically used measures of Plaque Burdens, it is within the scope of the present invention in those embodiments of the invention using a measure of Plaque Burden to use any number as a limit of boundary that exceeds about 55%.

The present invention has been described in connection with certain embodiments, combinations, configurations and relative dimensions. It is to be understood, however, that the description given herein has been given for the purpose of explaining and illustrating the invention and are not intended to limit the scope of the invention. In addition, it is clear than an almost infinite number of minor variations to the form and function of the disclosed invention could be made and also still be within the scope of the invention. Consequently, it is not intended that the invention be limited to the specific embodiments and variants of the invention disclosed. It is to be further understood that changes and modifications to the descriptions given herein will occur to those skilled in the art. Therefore, the scope of the invention should be limited only by the scope of the claims.

1. A method of determining whether a patient's physiology puts him or her at an increased risk of experiencing a Clinical Event comprising the steps of:
ascertaining, for one of more locations along an artery of interest, the Plaque Burden of the artery, the Minimum Lumen Area of the artery and whether a VH-TCFA is present;
determining whether there is a concurrence for values for the Plaque Burden being equal to or greater than about 70%, the Minimum Lumen Area being less than or equal to about 4 mm² and a VH-TCFA being present; and
communicating whether such concurrence has occurred.

2. A method of determining whether a patient's physiology puts him or her at an increased risk of experiencing a Silent Clinical Event comprising the steps of:
ascertaining, for one of more locations along an artery of interest, the Plaque Burden of the artery, the Minimum Lumen Area of the artery and whether a VH-TCFA is present;
determining whether there is a concurrence for values for the Plaque Burden being between about 30% and about 70%, the Minimum Lumen Area being less than or equal to about 4 mm² and multiple VH-TCFAs being present; and
communicating whether such concurrence has occurred.

3. A method of determining whether a patient's physiology puts him or her at an increased risk of experiencing a Clinical Event comprising the steps of:
ascertaining, for one of more locations along an artery of interest, whether two of the following parameters fall within selected targets: the Plaque Burden of the artery being equal to or greater than about 70%, the Minimum Lumen Area of the artery being less than or equal to about 4 mm² and whether a VH-TCFA is present;
determining, for the two ascertained parameters, whether the two selected parameters both fall within their respective selected targets; and
communicating whether the two selected parameters both fall within their respective selected targets.

4. The method of claim 4 wherein the selected parameters are the Plaque Burden of the artery and the Minimum Lumen Area of the artery.

5. The method of claim 4 wherein the selected parameters are the Plaque Burden of the artery and whether a VH-TCFA is present.

6. The method of claim 4 wherein the selected parameters are the Minimum Lumen Area of the artery and whether a VH-TCFA is present.

7. A method of determining whether a patient's physiology puts him or her at an increased risk of experiencing a Clinical Event comprising the steps of:
ascertaining, for one of more locations along an artery of interest, whether a parameter of interest falls within a selected target;
communicating whether the selected parameter falls within its selected target.

8. The method of claim 7 wherein the parameter of interest is the Plaque Burden and the selected target is the Plaque Burden being equal to or greater than about 70%.

9. The method of claim 7 wherein the parameter of interest is the Minimum Lumen Area of the artery and the selected target is the Minimum Lumen Area being less than or equal to about 4 mm².

10. The method of claim 7 wherein the parameter of interest and selected target is whether a VH-TCFA is present.

11. A device for determining whether a patient's physiology puts him or her at an increased risk of experiencing a Clinical Event comprising:
a tissue-characterization system;
a clinical outcome predicted device running according to the steps of:
ascertaining, for one of more locations along an artery of interest, the Plaque Burden of the artery, the Minimum Lumen Area of the artery and whether a VH-TCFA is present;
determining whether there is a concurrence for values for the Plaque Burden being equal to or greater than about 70%, the Minimum Lumen Area being less than or equal to about 4 mm² and a VH-TCFA being present; and
communicating whether such concurrence has occurred.

12. A device for determining whether a patient's physiology puts him or her at an increased risk of experiencing a Silent Clinical Event comprising the steps of:
a tissue-characterization system;
a clinical outcome predicted device running according to the steps of:
ascertaining, for one of more locations along an artery of interest, the Plaque Burden of the artery, the Minimum Lumen Area of the artery and whether a VH-TCFA is present;
determining whether there is a concurrence for values for the Plaque Burden being between about 30% and about 70%, the Minimum Lumen Area being less than or equal to about 4 mm² and multiple VH-TCFAs being present; and
communicating whether such concurrence has occurred.

13. A device for determining whether a patient's physiology puts him or her at an increased risk of experiencing a Clinical Event comprising the steps of:
a tissue-characterization system;
a clinical outcome predicted device running according to the steps of:
ascertaining, for one of more locations along an artery of interest, whether two of the following parameters fall within selected targets: the Plaque Burden of the artery being equal to or greater than about 70%, the Minimum Lumen Area of the artery being less than or equal to about 4 mm² and whether a VH-TCFA is present;
determining, for the two ascertained parameters, whether the two selected parameters both fall within their respective selected targets; and
communicating whether the two selected parameters both fall within their respective selected targets.

14. The device of claim 13 wherein the selected parameters are the Plaque Burden of the artery and the Minimum Lumen Area of the artery.

15. The method of claim 13 wherein the selected parameters are the Plaque Burden of the artery and whether a VH-TCFA is present.

16. The method of claim 14 wherein the selected parameters are the Minimum Lumen Area of the artery and whether a VH-TCFA is present.

17. A device for determining whether a patient's physiology puts him or her at an increased risk of experiencing a Clinical Event comprising the steps of:
a tissue-characterization system;
a clinical outcome predicted device running according to the steps of:
ascertaining, for one of more locations along an artery of interest, whether a parameter of interest falls within a selected target;
communicating whether the selected parameter falls within its selected target.

18. The method of claim 17 wherein the parameter of interest is the Plaque Burden and the selected target is the Plaque Burden being equal to or greater than about 70%.

19. The method of claim 17 wherein the parameter of interest is the Minimum Lumen Area of the artery and the selected target is the Minimum Lumen Area being less than or equal to about 4 mm².

20. The method of claim 17 wherein the parameter of interest and selected target is whether a VH-TCFA is present.
